(19) United States
(12) Patent Application Publication
(10) Pub. No.: US 2024/0017041 A1
(43) Pub. Date: Jan. 18, 2024

(54) STEERABLE CATHETER WITH FORCE SENSOR

(71) Applicant: Eindhoven Medical Robotics B.V., Eindhoven (NL)

(72) Inventors: Anupam Vasudeva Nayak, Eindhoven (NL); Rolf Iwan Gaasbeek, Eindhoven (NL)

(21) Appl. No.: 18/039,623

(22) PCT Filed: Dec. 10, 2021
(86) PCT No.: PCT/NL2021/050757

§ 371 Date: May 31, 2023

(30) Foreign Application Priority Data
Dec. 16, 2020 (NL) 2027126

(51) Int. Cl. A61M 25/01; A61F 11/20

(52) U.S. Cl. CPC A61M 25/0147; A61F 11/20; A61M 2025/015

(57) ABSTRACT

The invention deals with a steerable catheter system. Such systems are used in Minimal Invasive Systems (MIS) for getting access to difficult to reach places, like body cavities or blood vessels in for instance the human anatomy. The steerable catheter comprises a polymer shaft with a bending portion that comprises a pulling wire mechanically anchored at a distal part of the bending portion, where the shaft is provided with a central lumen and a force sensor at the distal part. According to the invention the pulling wire is the connecting wire to the force sensor and the pulling wire is mechanically anchored by a connector between the pulling wire and the force sensor.

Steerable catheter systems are used in Minimal Invasive Systems (MIS) for getting access to difficult to reach places, like body cavities or blood vessels in for instance the human anatomy. For that purpose the distal end of a catheter can bend, so that the catheter can be guided along a complicated route. The catheter comprises a central lumen (hole) that can be used for different purposes, like removing or inserting fluid, transporting a medical device, like electrodes, scissors or a camera (endoscope).

The invention deals with a steerable catheter comprising a polymer shaft with a bending portion that comprises a pulling wire mechanically anchored at a distal part of the bending portion, where the shaft is provided with a central lumen and a force sensor at the distal part.

Such a device is known from US 2011-0106101 A1. Here the catheter is a cochlear implant (CI) electrode. Cochlear implants are electrodes placed deep in the inner ear, the cochlea, to improve the hearing of a person. The cochlea has a spiral form. Such a CI electrode needs to be maneuvered through the cochlea to bring it in place. Any damage to the cochlea, in particular penetrating a membrane on the wall of the cochlea, the basilar membrane, should be prevented for a successful placement of the CI electrode. In many cases pre-curved (also called perimodiolar) CI electrodes are used. The known prior art uses a CI electrode comprising a polymer shaft with a bending portion that comprises in a central lumen pulling wires mechanically anchored at a distal part of the bending portion (called a ‘steerable stylet’ in the application). Such a CI electrode allows active control over the bend of the electrode, facilitating insertion of the CI electrode into the spirals of the cochlear. To prevent damage during insertion the known CI electrode is equipped with a force sensor. The sensor indicates when a wall of the cochlear is touched and allows steering away from the wall.

The known device has the disadvantage that the CI electrode becomes thicker because of the use of the force sensor and its connecting wire(s). This means that the known device cannot reach the deepest parts of an averaged size cochlea. Even if further developments allow the size to reduce, it is unlikely that people with small cochlea can be optimally treated with these type of devices.

It is the aim of the present invention to provide a steerable catheter with a force sensor that is able to reach through very small openings.

According to the invention the pulling wire is the connecting wire to the force sensor and the pulling wire is mechanically anchored by a connector between the pulling wire and the force sensor. This means that the force sensor is connected to the outside world via the pulling wire(s). Pulling wires need a mechanical anchoring to make it possible to bend the catheter. Here connectors between the force sensor and the pulling wires are used as mechanical anchors. As a force sensor different devices can be used. A possibility is the use of an optical force sensor, like a fibre Bragg sensor or Fabry-Perot sensor as in the prior art. Such sensors are connected via an optical fibre. This optical fibre is connected via a fibre connector to the optical sensor. This optical connecting wire (fibre) is used as the pulling wire. The optical connector then serves as the mechanical anchoring for the optical connector fibre (wire). The optical connector is larger in diameter than the optical fibre. Thus it can be used as mechanical anchoring for the optical connecting fibre, so that the fibre can be used as the pulling wire. Also an electrical force sensor like a strain gauge, for instance a silicon strain gauge, can be used as in the prior art. The strain gauge is connected by two connecting wires to measure a resistance change. In that case the pulling wires should then be made of an electrically conducting material, for instance a metal or a carbon fibre. In that case the connectors can be for instance metal rings or hollow cylinders, where on the one side the electrically conducting pulling wires are introduced an on the other side the connecting wires of the strain gauge. The wires can be (laser)welded or clamped in the ring or cylinder. Such a ring or cylinder has a larger radius than the pulling wires, and thus it will provide sufficient anchoring for the pulling wires.

The use of connecting wires for the force sensor as pulling wires enables a smaller catheter, since the connector wires have a double function. They serve both as pulling and as connecting wires, thus halving the amount of wires in the catheter. The inventive catheter can thus be made much smaller than a catheter according the prior art.

Thus the connectors have a larger outer diameter than the pulling wires and thus prevent slippage of the pulling wire into the polymer shaft, i.e. they provide anchoring for the pulling wires. This anchoring can be further improved when the connectors have radially extending protrusions. Such protrusions provide extra grip for the connectors on the polymer of the shaft, thus allowing larger forces on the pulling wires without causing slippage.

Preferably the pulling wires are electrically conducting and the force sensor comprises a Shape Memory Alloy (SMA) wire. The pulling wires are electrically conducting and at the distal part two pulling wires are electrically connected by a Shape Memory Alloy (SMA) wire that can serve as the force sensor. The invention uses the pulling wires as electrical conducts to a force sensor made of an SMA wire. This means that the resistance of the material of the pulling wires should not change when the material is put under stress, i.e. the pulling wires are being pulled. Such an SMA wire sensor can be made very small and by using the pulling wires as electrical conducts to the sensor the total size of the catheter remains very small. Thus, a catheter according to the invention can be inserted in very small openings.

The use of SMA wire in a force sensor is known from IEEE SENSORS JOURNAL, VOL. 17, NO. 4, Feb. 15, 2017: Shape Memory Alloy Wire for Force Sensing, Joshephine Selvarani Ruth D and K. Dhanalakshmi. In this paper an SMA wire is connected at an angle between the fixed and free ends of a cantilever beam. Also the use of SMA wire as a sensor in the servo of a DC motor is described. These are all fairly large constructions. There is no indication nor hint that SMA wire can be used as described in this invention.

Preferably the SMA wire comprises a semi-circle. This means the length of the SMA wire is π×(distance between the centers of the pulling wires)/2 is ˜1.5× this distance. This length of the SMA wire provides enough sensitivity for most applications. In order to increase the sensitivity of the force sensor in a favorable embodiment the length of SMA wire is at least twice the distance between the centers of the pulling wires. The SMA wire can then be used in different shapes like a larger loop or a coiled loop. The extra deformation of the longer SMA wire enhances the sensitivity of the force sensor. The semi-circle and the coil also introduce elastic stresses in the SMA wire. Such stresses also give a larger sensitivity of the SMA wire.

The catheter can be used as in the prior art, but with a smaller, more sensitive force sensor.

Preferably the pulling wires and the SMA wire are located in lumen outside the central lumen along an axis of the shaft at a radial position away from the center of the shaft. Thus, in contrast to the prior art, the central lumen is not used for the pulling wires and the force sensor, but the pulling wires and the SMA force sensor are integrated in the polymer shaft in further lumen. This frees up the larger central lumen for the transport of fluids or a medical device. The extra advantage is that a device smaller than the catheter itself can be transported/moved through the central lumen to reach even smaller cavities.

Preferably the central lumen can be used for the transport of an electrode for a cochlear implant. The catheter is then used to transport the CI electrode into the cochlea. The catheter thus acts as a steerable sheath for the CI electrode. The CI electrode can be pushed through the main central lumen. In the smallest section of the cochlea the CI electrode is pushed out of the central lumen further into the cochlea. The catheter sheath can then later be removed if required.

In some cases the catheter needs to be removed while the medical device transported through the central lumen stays in place. For instance when inserting a CI electrode, contact surfaces on the CI electrode need to make contact with nerve cells in the cochlea. This is not possible if the CI electrode is still in the central lumen surrounded by the polymer shaft. Preferably the shaft is provided with a region that extends along the shaft in an axial direction, where the distance between an inner surface of the central lumen and an outer surface of the region is less than 200 μm. The inner surface of the central lumen is formed by the wall of the lumen. The outer surface of the region is formed by an outer surface of the polymer shaft. Such region can be formed by a special shape of the central lumen in the axial direction or it can be formed by a axial groove in the outer surface of the polymer shaft. Preferably the shape of the central lumen or the axial groove contains a sharp edge where the stresses are large. Such a sharp edge serves to introduce a crack in the region that will propagate in the axial direction along the catheter. The region serves as a breaking line along the catheter, i.e. it forms a breaking point where the shaft can easily be split when a radial force is applied. Such a split facilitates removal of the catheter, while the medical device can be left in place.

In an advantageous embodiment the catheter has at least two sets of two pulling wires connected by SMA wires. The two sets of two pulling wires and the possibility to rotate the catheter at the proximal part, i.e. the base, enable easy movement of the distal part of the catheter in different directions. The two pulling wires connected to the same SMA wire are located parallel next to each-other. They basically bend the catheter in the same direction. This also halves the forces on the mechanical connection of the pulling wires at the distal part.

The invention also deals with a system for a steerable catheter with at least two sets of two pulling wires connected by SMA wires where when a change in resistance of an SMA wire is measured, pulling wires connected to an alternate SMA wire are pulled. Such a system can be part of an automated catheter guidance system, for instance connected to a robot.

### DESCRIPTION FIGURES

The invention is further explained with the help of the following drawing in which

FIG. 1 shows a radial cross-section of a catheter according to the invention,

FIG. 2 shows an axial cross-section of a catheter at the distal part according to the invention along line A-A′ of FIG. 1,

FIG. 3 shows an axial cross-section of a further embodiment of the SMA sensor,

FIG. 4 shows a top view on the distal part of the catheter in the axial direction of a further embodiment of the catheter of the invention,

FIG. 5 shows an axial cross-section of a catheter according to the invention along line B-B′ of FIG. 4, FIG. 5 differs from FIG. 2 in the addition of a supporting cylinder,

FIG. 6 shows a radial cross-section of a further catheter according to the invention.

Directions are indicated with the shaft as a reference. Figures are for reference only and are not drawn to scale.

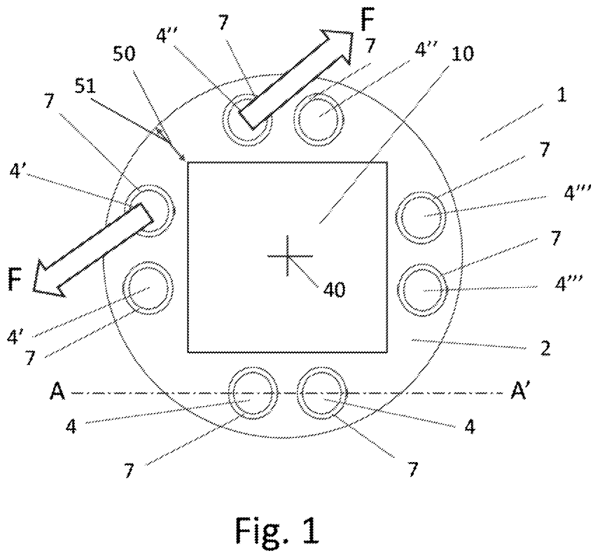

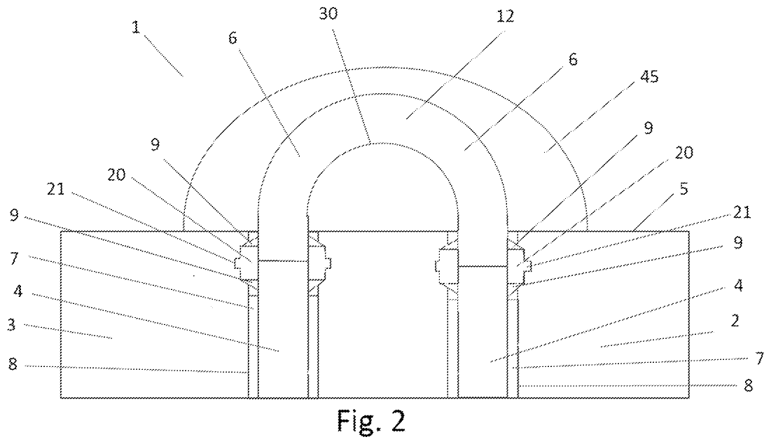

FIGS. 1 and 2 show a steerable catheter 1 comprising a polymer shaft 2 with a bending portion 3 that comprises pulling wires 4, 4′, 4″, 4′″ mechanically anchored at a distal part 5 of the bending portion 3, where the shaft 2 is provided with a central lumen 10 and a force sensor 12 at the distal part 5. Since the construction is the same for all wires 4, 4′, 4″, 4′″, only the construction of wires 4 will be discussed. The pulling wires 4 are connecting wires to the force sensor 12 and the pulling wires 4 are mechanically anchored by connectors 20 between the pulling wires 4 and the force sensor 12. This means that the force sensor 12 is connected to the outside world via the pulling wires 4. The pulling wires 4 need a mechanical anchoring to make it possible to bend the catheter 1. Here the connectors 20 between the force sensor 12 and the pulling wires 4 are used as mechanical anchors. As a force sensor 12 different devices can be used. As an electrical force sensor a strain gauge, for instance a silicon strain gauge, can be used as in the prior art. The pulling wires 4 should then be made of an electrically conducting material, for instance a metal or a carbon fibre. In that case the connectors 20 can be for instance metal rings or hollow cylinders, where on the one side the electrically conducting pulling wires 4 are introduced and on the other side the connecting wires of the strain gauge. The wires can be (laser)welded or clamped in the ring or cylinder 20. Such a ring or cylinder has a larger radius than the pulling wires 4, and thus it will provide sufficient anchoring for the pulling wires 4. Another possibility is the use of an optical force sensor, like a fibre Bragg sensor, Fabry-Perot sensor or an optical sensor that uses the change in light reflection from a mirror membrane. Such sensors are connected via one optical fibre. Optical fibres can be obtained in a range of diameters. A diameter between 50-75 μm is very suitable as connecting and pulling optical wire. This optical fibre is connected via a standard connector to the optical sensor as shown in the prior art. Also this optical connector can be used as the mechanical anchoring for the optical connector fibre (wire). Also in this case the connector is larger in diameter than the optical fibre. Thus the optical connector can be used as mechanical anchoring for the optical connecting fibre that is also functioning as the pulling wire. With an optical force sensor only one connecting wire (fibre) is necessary. The anchoring can be further improved when the connectors 20 have radially extending protrusions 21. Such protrusions 21 provide extra grip for the connectors 20 on the polymer of the shaft 2, thus allowing larger forces on the pulling wires 4 without causing slippage.

Preferably the pulling wires 4 are electrically conducting and the force sensor 12 comprises a Shape Memory Alloy (SMA) wire 6. The pulling wires 4 are electrically conducting and at the distal part 5 two pulling wires 4 are electrically connected by a Shape Memory Alloy (SMA) wire 6 that can serve as the force sensor 12. The invention uses the pulling wires 4 as electrical conducts to the force sensor 12 made of an SMA wire 6. This means that the resistance of the material of the pulling wires 4 should not change when the material is put under stress, i.e. the pulling wires are being pulled. Such an SMA wire sensor can be made very small and by using the pulling wires as electrical conducts to the sensor the total size of the catheter remains very small. Thus, a catheter according to the invention can be inserted in very small openings.

The SMA wire 6 can be used as a force/touch sensor 12 in the catheter 1. The SMA wire 6 is embedded in the distal part 5, i.e. the tip of the catheter 1. When the SMA wire 6 makes contact with a wall for instance of a body cavity, like a cochlear, the stress in the SMA wire 6 changes, causing the crystallographic structure to change. The air in-between the wall of the cavity and the SMA wire 6 is an insulator. Direct contact between SMA wire 6 and wall will cause an increase in temperature in the SMA wire. The latter further aids the transformation of the crystallographic structure of the SMA wire 6. These effects combined make the SMA wire's resistivity very sensitive to wall contact.

The resistivity change in the SMA wire 6 is measured by applying a small current through the SMA wire 6. The electrically conducting pulling wires 4 are used both for pulling, as well as delivering the current through the SMA wire 6.

In this example the diameter of the shaft 2 is 2 Fr (0.67 mm). The eight pulling wires 4 can be made of any conducting material that has enough strength to withstand the pulling forces and does not change resistivity when under stress. Such materials could be for instance titanium or a carbon fibre. In this example stainless steel wires with a diameter of 50 μm are used as pulling wires 4. The pulling wires are surrounded by a PTFE liner 7 with a thickness of 20 μm. The pulling wires 4 and the PTFE liner 7 are located in lumen 8 with a diameter of 95 μm. The diameter of the SMA wire 6 is the same as that of the pulling wire: 50 μm. The SMA wire 6 is made of NiTi (Nitinol). The bending portion 3 of the shaft 2 is made of a copolymer of polyether and polyamide with a Young's modulus of 18 MPa, a yield stress of less than 4 MPa and a melting point of 144° C. The length of the shaft 2 in the axial direction is 115 mm. The shaft 2 can be made of sections with different stiffness, for instance a stiffer part near the proximal part of the catheter 1 can be made of a copolymer of polyether and polyamide with a Young's modulus of 510 MPa, a yield stress of 26 MPa and a melting point of 174° C. Such a stiffer part makes easier manipulation of the catheter, like introducing a rotation, possible. The central lumen 10 is a square hole with sides of 370 μm.

Of course the catheter can be made with different dimensions. For instance the total diameter of the shaft 2 can be made much smaller when the central lumen 10 is reduced in size or shape. Also when the number of sets of pulling wires 4 is reduced to for instance two or three the overall outer dimensions of the catheter 1 can be reduced considerably. When using optical force sensors only one optical fibre is necessary for connecting the sensor. Three optical fibres connected to optical force sensors then make a very small diameter catheter.

FIG. 2 shows how the pulling wires 4 are mechanically anchored by connectors 20 between the SMA wire 6 and the pulling wires 4. The connectors 20 can be for instance metal rings. The SMA wires 6 and a pulling wire 4 are then fixed inside the rings 20. Optimal performance, i.e. control of a bend of the tip of the catheter 1, is obtained when the anchoring is as close as possible to the distal part 5. Suitable materials for the connectors 20 are nickel or stainless steel. Laser-welding of the pulling wires 4 and the SMA wire 6 to the connectors 20 then provides an excellent, strong and electrically conducting bond between the pulling wires 4 and the SMA wire 6. FIG. 2 shows welded regions 9 on both sides of the connector 20. The thickness of the connector rings 20 of FIG. 2 is 20 μm with a length of 500 μm. An alternative approach is to clamp a nickel cylinder 20 over the interface 25 between the pulling wire 4 and the SMA wire 6. The clamping length required for a robust fixation with a clamped cylinder is inherently larger (>1000 μm) as compared to the length required for a ring 20 plus laser-welding 9 solution, resulting in a larger part of the shaft 2 that bends less easy. Thus using a ring 20 and laser-welding 9 is the preferred approach to the connectors 20. The connectors 20 have a larger outer diameter than the SMA wire 6 and the pulling wires 4 and thus prevent slippage of the pulling wire 4 into lumen 8 of the polymer shaft 2, i.e. they provide anchoring for the pulling wires 4. Note that after manufacturing as described further the lumen 8 will be smaller than when assembly starts. This anchoring can be further improved when the connectors 20 have radially extending protrusions 21. In this example the protrusions extend 10 μm from the connectors 20. Such protrusions 21 provide extra grip for the connectors 20 on the polymer of the shaft 2, thus allowing larger forces on the pulling wires 4 without causing slippage.

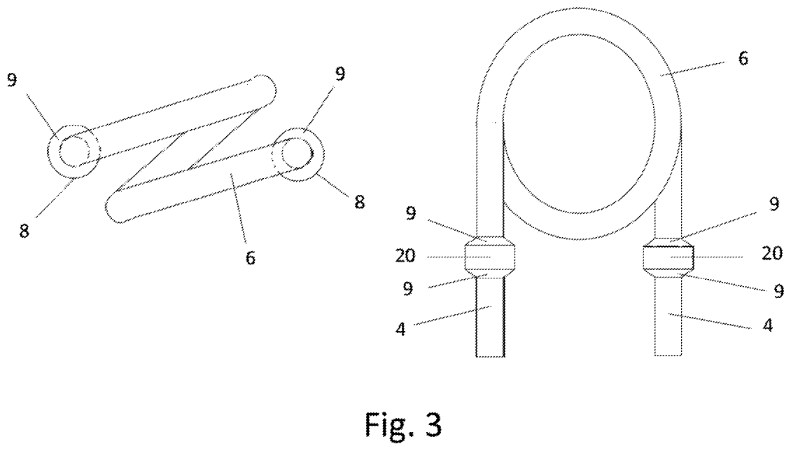

FIG. 2 shows that preferably the SMA wire comprises a semi-circle 30. The centers of the lumen 8 in this embodiment are located 115 μm apart. This means the length of the SMA wire is π×(distance between the centers of the pulling wires)/2 is ˜1.5×115 μm is 175 μm. This length of the SMA wire 6 provides enough sensitivity for most applications. Moreover it is easy to manufacture such a semicircle 30. In order to increase the sensitivity of the force sensor 12 in a favorable embodiment the length of SMA wire 6 is at least twice the distance between the centers of the pulling wires 4. FIG. 3 shows an example of an SMA wire 6 and pulling wires 4 that has a larger length. The example of FIG. 3 shows the SMA wire 6 just before insertion in the lumen 8. FIG. 3 does not show the polymer shaft 2. In this case the length of the SMA wire 6 is increased by creating a coil in the SMA wire 6. The larger length of the SMA wire 6 increases the sensitivity of the force sensor. Bending the SMA wire 6 in a semi-circle or coil gives elastic stresses in the SMA wire 6: tensile at the outer side of the bend and compressive at the inner side of the bend in the wire 6. Especially the tensile stresses give an extra increase in the sensitivity of the SMA sensor.

For a 175 μm SMA wire of 75 μm diameter in a semi-circle, a full loop (so full transformation) results in a 265 μΩ change in resistance of the SMA wire 6. For a 175 μm SMA wire of 50 um diameter in a semi-circle, a full loop (so full transformation) results in a 175 μΩ change in resistance of the SMA wire 6.

The catheter 1 can be used as in the prior art, but with a smaller, more sensitive force sensor 12. FIGS. 1, 4 and 6 show that preferably the pulling wires 4 and the SMA wire 6 are located in lumen 8 outside the central lumen 10 along an axis of the shaft 2 at a radial position away from the central axis 40 of the shaft 2. Thus the central lumen is not used for the pulling wires 4 and the force sensor 12, but the pulling wires 4 and the SMA force sensor 12 are integrated in the polymer shaft 2 in lumen 8. This frees up the larger central lumen 10 for the transport of fluids or a medical device. The extra advantage is that a device smaller than the catheter 1 itself can be transported/moved through the central lumen 10 to reach even smaller cavities. A lubricant, like parylene, can be applied to the central lumen 10 to ease the transport of the medical device.

The catheter 1 is manufactured as follows. The polymer shaft 2 is made via extrusion. That way the polymer is shaped with the lumen 8 for the pulling wires and the central lumen 10. As mentioned the shaft 2 can have different stiff and more bendable parts, made by using different polymers. The stiff and the more bendable part have the same shape. The connector rings 20 are made either by 3D metal printing or by deformation, etching or stamping of a stainless steel pellet. The SMA wire 6 is cut to the desired length and then laser-welded onto pulling wires 4 using the connector rings 20, thus providing the laser-welded regions 9 around the rings 20. The pulling wires 4 are provided with a PTFE liner 7. Then the pulling wires 4 with liner 7 and the connected SMA wires 6 are introduced in the lumen 8. The liners 7 do not reach all the way up to the connecting rings 20, but there is a blank area with a length of approximately 50 μm between the top of the liner 7 and the rings 20. The shaft is then surrounded by a temporary heat shrink tube that has a shrinking temperature of 220° C. The temporary heat shrink is not critical as long as it provides a rather large shrinkage at this temperature. A mandrel is placed in the central lumen 10 to prevent shrinkage of this lumen 10. The complete structure is then heated to 220 degrees Celsius, while a relatively high preload (˜25N) is applied to the pulling wires 4 at the proximal part of the catheter 1. The temporary heat shrink tube and the high temperature melt the polymer of the shaft 2 and provide strong inwards pointing radial forces on the shaft 2. This removes all slack between the lumen 8, the liners 7 and the pulling wires 4. The preload causes the connectors 20, 21 to sink partly into the lumen 8 during the melting because there is the blank region without the PTFE liner 7. The preload also provides a prestress in the SMA wire 6. After cooling to room temperature the temporary heat shrink tube and the mandrel are removed. The pulling wires 4 can still easily slide in the lumen 8 because of the PTFE linings 8. The mandrel makes sure the dimensions of the central lumen 10 do not change. The stiff and more bendable parts of the shaft 2 also melt together and the connectors 20 and protrusions 21 if provided, are pushed by the radial forces in the molten polymer of shaft 2. Thus the connectors 20, 21 provide a very good anchoring point for the pulling wires 4. The SMA wire 6, i.e. the force sensor 12 is then covered with a coating 45 made of a medical device adhesive, such as Dymax 1072, applied with a thickness of 100 μm. The total diameter of shaft 2 after manufacturing is approximately 0.55 μm. The shaft can also be covered by a tube made of a medical grade polymer if desired.

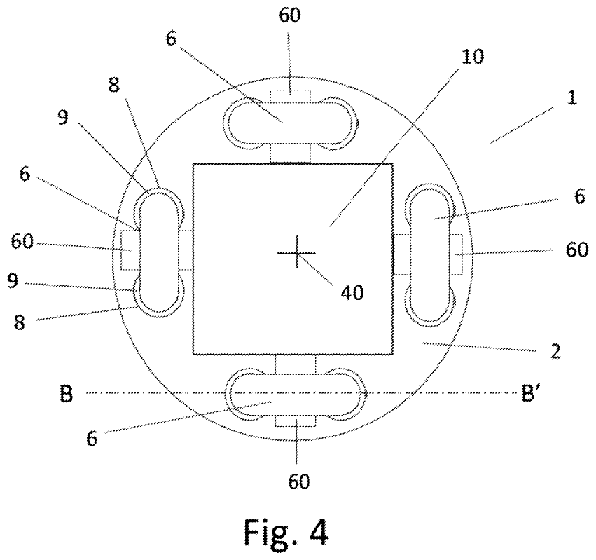

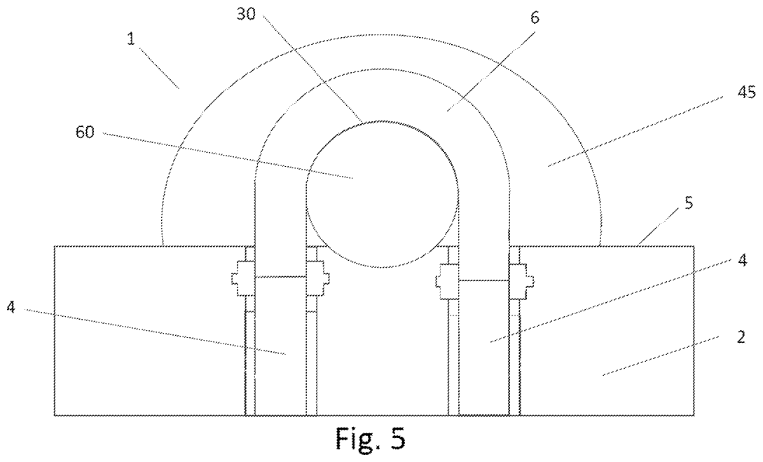

If possible the SMA wire 6 is provided with a prestress of about 100 Mpa. Such a prestress further increases the sensitivity of the SMA wire 6. Such a prestress is introduced when making the catheter 1. FIGS. 4 and 5 show how such a prestress can be introduced more precisely during manufacturing. FIGS. 4 and 5 show how a cylinder 60 can be used as a support for the semi-circle 30 of the SMA wire 6. The cylinder 60 can be easily made from a parts of a strong non-conducting wire, like a polyimide polymer wire. Polyimide has a Young's modulus of 2.5 GPa, a yield stress of 230 MPa and a melting temperature of 375° C. The polyimide cylinder 60 supports the semicircle SMA wire 6. The polyimide has a higher melting temperature than the temperature applied during the heat shrink. Thus it will maintain it shape and supports the SMA wire 6. When during the heating step for the heat shrink the pulling wires 4 are loaded with a force of ˜25N, the connectors 20 will sink in the lumen 8, but the cylinder 60 will further anchor the SMA wire, thus introducing a prestress of about 100 MPa. The cylinder 60 can also be used to make the coil described in FIG. 3 by winding the SMA wire around the cylinder 60. If need be the cylinder 60 can be removed after the heat treatment.

Preferably the central lumen 10 can be used for the transport of an electrode for a cochlear implant (CI). The catheter 1 is then used to transport the CI electrode into the cochlea. The catheter 1 thus acts as a steerable sheath for the CI electrode. The CI electrode can be pushed through the main central lumen 10. In the smallest section of the cochlea the CI electrode is pushed out of the central lumen 10 and thus out of the catheter 1 further into the cochlea. The catheter sheath 1 can then later be removed if required.

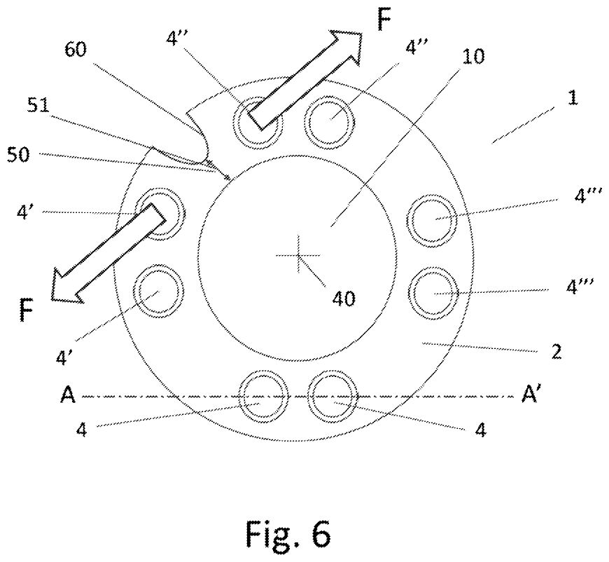

In some cases the catheter 1 needs to be removed while the medical device transported through the central lumen stays in place. For instance when inserting a CI electrode, contact surfaces on the CI electrode need to make contact with nerve cells in the cochlea. This is not possible if the CI electrode is still in the central lumen 10 surrounded by the polymer shaft 2. FIGS. 1 and 6 show how preferably the shaft 2 is provided with a region 50 that extends along the shaft 2 in the axial direction, where the distance 51 between an inner surface of the central lumen 10 and an outer surface of the region is less than 200 μm. The inner surface of the central lumen 10 is formed by the wall of the lumen 10. The outer surface of the region 50 is formed by an outer surface of the polymer shaft 2. FIG. 1 shows that such a region 50 can be formed by a special shape of the central lumen 10. The square shape of lumen 10 in FIG. 1 has relatively sharp corners, where this distance 51 is short. FIG. 6 shows that as an alternative the distance 51 can also be created by providing the outer surface of the shaft 2 with a groove 60. This groove 60 should preferably have a relatively sharp point at the bottom of the groove 60. The region 50 serves as a breaking line along the catheter 1, i.e. it forms a breaking point where the shaft 1 can easily be split when forces F as shown if FIGS. 1 and 6 are applied. The forces F can be applied via the pulling wires 4. Such a split facilitates removal of the catheter 1, while the medical device can be left in place. The catheter 1 is zipped open, while pulling the catheter 1 out and leaving the medical device behind.

In an advantageous embodiment the catheter 1 has at least two sets of two pulling wires 4 connected by SMA wires 6. The two sets of two pulling wires 4 and the possibility to rotate the catheter 1 at the proximal part, i.e. the base, enable easy movement of the distal part 5 of the catheter 1 in different directions. In the example the two pulling wires 4 connected to the same SMA wire 6 are located parallel next to each-other. They basically bend the catheter 1 in the same direction. This also halves the forces on the mechanical anchoring connection 20 of the pulling wires at the distal part 5.

It is also possible to have one optical fibre pulling wire asymmetric from the central axis of the catheter and then use this optical fibre as the connecting wire to an optical sensor. By rotating the catheter at the basis, i.e. the proximal part of catheter 1, and using the pulling wire to get the bend in the required direction this type of catheter can be very useful to introduce a CI electrode in the cochlea. Such a catheter with only a single pulling wire and an optical sensor can be made very small. As an alternative one set, i.e. two parallel electrically conducting pulling wires 4 close to each other in an asymmetric position can be used to make a very small similar catheter with an electric force sensor 12.

The invention also deals with a system for a steerable catheter 1 with at least two sets of two pulling wires 4 connected by SMA wires 6 where when a change in resistance of an SMA wire 6 is measured, pulling wires 4 connected to an alternate SMA wire 6 are pulled.

The catheter would be operated by an operator. The operator in this case could be a (medical) specialist and/or an automated system (e.g., robot). Input actions would be insertion of the medical device in the central lumen 10, rotation action of the shaft 2 and/or pulling the pulling wires to steer. The actions can be facilitated with a user-interface/joystick, possibly with additional information (e.g. navigation tools with overlay of medical images, etc.). Further, to allow the touch sensing when using an SMA wire 6 as force sensor, an ancillary device should send a small current through the sets of pulling wires 4 and measure resistivity changes (caused by crystallographic structure changes in the SMA). An identical setup would send light through an optical fibre to measure changes introduced by an optical sensor.

1. A steerable catheter comprising a polymer shaft with a bending portion that comprises a pulling wire mechanically anchored at a distal part of the bending portion, where the shaft is provided with a central lumen and a force sensor at the distal part, characterized in that the pulling wire is the connecting wire to the force sensor and the pulling wire is mechanically anchored by a connector between the pulling wire and the force sensor.

2. The steerable catheter according to claim 1, characterized in that the connector has a radially extending protrusion.

3. The steerable catheter according to claim 1, characterized in that two pulling wires are electrically conducting and the force sensor comprises a Shape Memory Alloy (SMA) wire, where the two pulling wires connect to different ends of the SMA wire.

4. The steerable catheter according to claim 3, characterized in that the SMA wire comprises a semi-circle.

5. The steerable catheter according to claim 3, characterized in that the length of SMA wire is at least twice the distance between the centers of the pulling wires.

6. The steerable catheter according to claim 1, characterized in that pulling wires and the force sensor are located in lumen outside the central lumen along an axis of the shaft at a radial position away from the center of the shaft.

7. The steerable catheter according to claim 6, characterized in that the central lumen can be used for the transport of an electrode for a cochlear implant.

8. The steerable catheter according to claim 6, characterized in that the shaft is provided with a region that extends along the shaft in an axial direction, where the distance between an inner surface of the central lumen and an outer surface of the region is less than 200 μm.

9. The steerable catheter according to claim 3, characterized in that the catheter has at least two sets of two pulling wires connected by SMA wires.

10. A system for a steerable catheter according to claim 9, characterized in that when a change in resistance of an SMA wire is measured, pulling wires connected to an alternate SMA wire are pulled.
